(19) United States
(12) Patent Application Publication
(10) Pub. No.: US 2006/0096870 A1
(43) Pub. Date: May 11, 2006

(54) Detection of biological molecules

(76) Inventors: Fwu-Shan Sheu, Singapore (SG); Jianshan Ye, Singapore (SG)

Correspondence Address: YOUNG & THOMPSON, 745 SOUTH 23RD STREET, 2ND FLOOR, ARLINGTON, VA, 22202, US

(21) Appl. No.: 10/984,890

(22) Filed: Nov. 10, 2004

(51) Int. Cl. G01N 27/26; G01N 33/487; G01F 1/64

(52) U.S. Cl. 205/775; 205/792; 204/403.01; 204/400

(57) ABSTRACT

An apparatus uses carbon nanotubes for electrochemical analysis of biological molecules of interest such as Uric Acid, illustrating how the voltammetric behaviors of uric acid (UA) and L-ascorbic acid (L-AA) at a well-aligned, carbon nanotube, electrode may be used in a biochemical assay. Compared to glassy carbon, a carbon nanotube electrode reduces troublesome overpotentials. Based on its differential catalytic function toward the oxidation of UA and L-AA, the carbon nanotube electrode can be used for a selective determination of UA in the presence of L-AA. The peak current obtained from DPV was linearly dependent on the UA concentration in the range of 0.2 μM to 80 μM with a correlation coefficient of 0.997. The detection limit (3δ) for UA was found to be 0.1 μM. The device allows for detection of UA in a human urine sample, even in the presence of high concentrations of L-AA, using only simple dilution.

### 1. FIELD OF THE INVENTION

This invention relates to an apparatus and a method for detecting the presence of a molecule in a sample. Preferably, but not exclusively, the apparatus and method are used to selectively detect the presence of one or more biological molecules such as organic acids, sugars, proteins, hormones, cofactors and amino acids and more particularly the presence of Uric Acid (UA) in a sample of a biological fluid. Most particularly, the invention relates to a method of detecting the presence of Uric Acid in a urine sample, which also contains molecules having similar oxidation potentials such as L-ascorbic acid (L-AA).

### 2. BACKGROUND OF THE INVENTION

Uric acid (UA) is a very important biological molecule present in body fluids. Extreme abnormalities of UA levels are symptoms of several diseases such as pneumonia, fatal poisoning with chloroform or methanol, or toxemia during pregnancy [1]. In general, electro-active UA can be irreversibly oxidized in aqueous solution and the major product is allantoin [2-3]. As UA and ascorbic acid (L-AA) are co-present in biological fluids such as blood and urine, it is important to develop a technique to selectively detect UA in the presence of L-AA conveniently in a routine assay. However, the direct electro-oxidation of UA and L-AA at bare electrodes requires high overpotentials [4], and UA and L-AA can be oxidized at a very similar potential [5], which results in rather poor selective detection. Thus, the ability to be able to selectively determine UA and L-AA has become a major goal of electro-analytical research [6, 7]. In particular, the basal concentration of UA and L-AA in biological samples varies from species to species in a wide range from 1.0×10⁻⁷ to 1.0×10⁻³ mol/L [8]. Therefore both sensitivity and selectivity are of equal importance in developing voltammetric procedures for biological detection. Various approaches have been attempted to solve the problems, which included ion-exchange membrane-coating [9-10], chemical [11-16] and/or enzyme-based [17-19] modification of electrodes. Unfortunately, long-term stability is hardly achieved by any enzyme-based method although tedious modification processes are involved. Up to now, sensitive and selective methods are still to be developed for the detection of UA due to its clinical significance.

Carbon nanotubes, consisting of cylindrical graphene sheets with nanometer diameter, possess in a unique way with high electrical conductivity, high chemical stability, and extremely high mechanical strength and modulus [20]. These special properties of both single-walled and multi-walled carbon nanotubes (SWNTs and MWNTs, respectively) have attracted increasing attention. Carbon nanotubes behave electrically either as metals or as semiconductors, depending on the architecture of the atomic structure [21-23]. The subtle electronic properties suggest that carbon nanotubes have the ability to promote electron-transfer reactions when used as an electrode material in electrochemical reactions, representing a new application of carbon nanotubes. The better performance of the carbon nanotube electrodes in comparison with other forms of carbon electrode has been attributed to the carbon nanotube dimensions, the electronic structure, and the topological defects present on the tube surface [24]. Furthermore, it has been proved that carbon nanotubes have better conductivity than graphite [25].

There have been some important works about the application of carbon nanotubes in electro-catalysis [24-29] and chemical sensors [26, 30, 31]. To construct a carbon nanotube electrode, SWNTs are shaped into an electrode by filtering suspension of nanotubes on a membrane filter [32] to form a paper of nanotube. Another method is casting the SWNTs suspension at the surface of solid electrodes such as Pt, Au, or glassy carbon [33-35]. However, for construction of a MWNTs electrode, the MWNTs are usually mixed with bromoform [24], mineral oil [27], or packed into the cavity at the tip of a microelectrode to form a carbon nanotube powder microelectrode [36]. MWNTs electrodes prepared by these methods may suffer from mechanical instability during detection, thus limiting their practical application. Fortunately, high-density well-aligned carbon nanotubes, which are multi-walled and vertically aligned on a large area of substrates, have been synthesized [37, 38]. These carbon nanotubes aligned on the substrate are very stable and can be used directly as electrochemical sensors. In this work, the selective detection of UA in the presence of high concentration of L-AA by carbon nanotube electrode is clearly demonstrated. In addition, MWNTs electrode shows good sensitivity and selectivity for the determination of UA in human urine samples.

Surprisingly, the inventors have found an alternative voltammetric method and apparatus for measuring the voltammetric profiles of biological molecules of special interest and are especially useful as a means for detecting and/or measuring important biological molecules such as Uric Acid and L-Ascorbic Acid. The inventors have also found that individual biological species which were previously difficult or impossible to distinguish when together in a sample can now be identified and/or measured. As such, the method of the invention may be used in diagnostic, analytical, and forensic tests. The inventors have also discovered the hitherto before unknown use of a novel electrode system to test biological fluids without the need for extensive and time-consuming pre-testing workup regimes. In particular, the inventors have developed a method and apparatus which allows the testing of biological fluids such as urine samples for the presence of Uric Acid even when in admixture with L-Ascorbic Acid and wherein the biological fluids do not require any further pretreatment other than a standard dilution step.

### 3. OBJECT OF THE INVENTION

It is an object of the invention to provide an improved method and apparatus for detecting the presence of a molecule of interest in a sample, or which will obviate or minimize the aforementioned disadvantages, or which will at least provide the public with a useful choice.

### 4. STATEMENTS OF THE INVENTION

Accordingly, in its broadest aspect the invention provides an electrochemical method for detecting the presence of one or more molecules of interest in a sample, characterized in that the method comprises a voltammetric analysis step using as a working electrode, a carbon nanotube electrode, a glassy carbon electrode or a Ta substrate electrode.

According to a second aspect of the invention there is provided an electrochemical method for selectively detecting the presence of one or more molecules of interest from a biological sample, characterized in that the method comprises a voltammetric analysis step using a carbon nanotube electrode as a working electrode.

Preferably, the molecule of interest is one or more of an organic acid, a sugar, a protein, a hormone, a cofactor, a vitamin, an amino acid or similar.

Preferably, the working electrode comprises a plurality of carbon nanotubes.

Preferably, the plurality of carbon nanotubes comprising the working electrode is/are high-density, multi-walled, well-aligned carbon nanotubes (MWNT's).

Preferably, the MWNT's are vertically aligned on a substrate.

Preferably, the substrate is selected from Ta, Co, Ni, V, Nb, Db, Pd, W, Mo, Cu, Fe, Si, Au, Pt, stainless steel, glassy carbon, graphite and diamond, or similar support.

Preferably, the high-density, multi-walled, well-aligned carbon nanotubes aligned on the substrate act as electrochemical sensors.

Preferably, the voltammetric analysis allows for the selective detection of Uric Acid (UA) in the presence of high concentration of L-Ascorbic Acid (L-AA) in a sample of interest.

Preferably, the voltammetric analysis allows for a voltammetric measurement in a cyclic voltammetry mode or a differential pulse voltammetry mode.

Preferably, the differential pulse voltammetry mode employs an increased potential of about 4 mV, a pulse amplitude of about 5 mV, a pulse period of about 0.2 s and a pulse width of about 0.05 s.

Preferably, the sample of interest is a biological fluid.

Preferably, the biological fluid is blood, urine, sweat, saliva, plasma, spinal fluid, a tissue culture, brain tissue, or similar.

Preferably, the biological fluid is a human blood or a human urine sample prepared by a simple dilution without the need for further pretreatment.

Preferably, the biological molecule of interest is Uric Acid and/or L-Ascorbic Acid.

Preferably, the one or more carbon electrodes act as electrochemical sensors sensitive to the presence of Uric Acid and L-Ascorbic Acid.

Preferably, the concentration of the molecule of interest is from 1.0×10⁻¹ to 1.0×10⁻⁹ mol/L, and more preferably from 1.0×10⁻³ to 1.0×10⁻⁷ mol/L

According to a third aspect the invention there is provided an apparatus for use in a method for electrochemically detecting the presence of one or more molecules of interest in a sample, characterized in that the apparatus comprises as a working electrode, a carbon nanotube electrode, a glassy carbon electrode or a Ta substrate electrode.

Preferably, the method is used as all or part of a diagnostic, analytical, or forensic test.

According to a fourth aspect of the invention there is provided an electrochemical apparatus for selectively detecting the presence of one or more molecules of interest from a biological sample, characterized in that the apparatus comprises a carbon nanotube electrode as a working electrode.

Preferably, the working electrode comprises high-density, multi-walled, well-aligned carbon nanotubes (MWNT's).

Preferably, the working electrode comprises high-density, multi-walled, well-aligned carbon nanotubes (MWNT's) connected to a glassy electrode via a Ta substrate.

Preferably, the apparatus comprises a three-electrode arrangement comprising as working electrode a MWNT's electrode, a platinum counter electrode and a 1M KCl—Ag|AgCl reference electrode.

A fifth aspect of the invention relates to a multi-walled carbon nanotube (MWNT).

A sixth aspect of the invention relates to the use a multi-walled carbon nanotube (MWNT) in a diagnostic, analytical, or forensic method.

A seventh aspect of the invention relates to an electrode suitable for use in an electro-analytical apparatus wherein the electrode comprises one or more multi-walled carbon nanotubes.

An eighth aspect of the invention relates to an electrode suitable for use in an electro-analytical apparatus wherein, the electrode comprises one or more high-density, multi-walled, well-aligned, carbon nanotubes as hereinbefore described in the Example and with reference to FIG. 1.

### 5. BRIEF DESCRIPTION OF THE INVENTION

This invention provides a method and apparatus for selectively testing for one or more biological molecules from a sample of biological material containing a mixture of similar biological molecules. Surprisingly, the inventors have found that carbon nanotubes can be used as an electrochemical apparatus for measuring the voltammetric profiles of biological molecules of special interest and are especially useful as a means of detecting and/or measuring important biological molecules such as Uric Acid and L-Ascorbic Acid. The inventors have also found that individual biological species which were previously difficult or impossible to distinguish when together in a sample can now be identified and/or measured. The inventors have also developed the hitherto before unknown use of multi-walled carbon nanotube (MWNT) electrodes to test biological fluids such as urine samples for the presence of Uric Acid even when the Uric Acid is in admixture with L-Ascorbic Acid and wherein the biological fluids do not require any further pretreatment other than a standard dilution step.

### 6. DESCRIPTION OF THE DRAWINGS

The invention will now be described by way of a non-limiting example only and with reference to the accompanying drawings in which:

FIG. 1. Scanning electron microscopy (SEM) image of the well-aligned MWNTs.

FIG. 2. Cyclic voltammograms of (a) the MWNTs electrode (solid line), (b) the glassy carbon electrode (dashed line), and (c) the Ta substrate electrode (dotted line) in pH 7.4 PBS. Scan rate, 50 mV·s⁻¹.

FIG. 3. Cyclic voltammograms of (A) 1 mM UA and (B) 1 mM L-AA in pH 7.4 PBS by using (a) the MWNTs electrode (solid line) or (b) the glassy carbon electrode (dotted line). Scan rate, 50 mV·s⁻¹.

FIG. 4. (A) Cyclic voltammograms of 0.5 mM UA and 0.5 mM L-AA mixture in pH 7.4 PBS by using (a) the MWNTs electrode (solid line) or (b) the glassy carbon electrode (dotted line). Scan rate, 50 mV·s⁻¹. (B) DPV responses of 0.5 mM UA and 0.5 mM L-AA mixture in pH 7.4 PBS by using (a) the MWNTs electrode (solid line) or (b) the glassy carbon electrode (dotted line). Parameters for DPV see text for details.

FIG. 5. pH dependence of (A) peak potentials and (B) peak currents at the MWNTs for UA (solid line) and L-AA (dotted line). Data of peak potentials and peak currents were taken from DPV response of 5.0 μM UA and 100 μM L-AA.

FIG. 6. DPV response of 5.0 μM UA at the MWNTs electrode in pH 7.4 PBS with the addition of (a) 0, (b) 50, (c) 100, (d) 200, (e) 300 and (f) 400 μM L-AA.

FIG. 7. DPV response of (a) 0, (b) 0.5, (c) 1.0, (d) 2.0 and (e) 4.0 μM UA in pH 7.4 PBS at the MWNTs electrode.

FIG. 8. Typical DPV response of (a) blank PBS and urine sample diluted (b) 500 times, (c) 250 times, and (d) 250 times with addition of 2.0 μM UA at the MWNTs electrode in pH 7.4 PBS.

FIG. 9. Cross Section view of the vertical-aligned multi-walled carbon nanotube electrode

FIG. 10. Schematic diagram of the apparatus for the detection of a biological solution by a carbon nanotube electrode.

### 7. DETAILED DESCRIPTION OF THE PREFERRED EMBODIMENTS OF THE INVENTION

The following non-limiting example is illustrative of a preferred method of working the Invention. Those of ordinary skill in the art will realize that integers used in the method will vary depending on both the sample to be tested and the molecule of interest to be detected. The inventors contemplate the method and apparatus will have use as part or whole of a diagnostic, analytical or forensic test. Qualitative and/or quantitative measurements may also be made.

### EXAMPLE

7.1. Chemicals and Reagents

UA and L-AA were obtained from Aldrich and were used as received. All other chemicals used were of reagent grade. Deionized water was obtained by purification through a Millipore water system and was used throughout. All solutions were freshly prepared daily.

7.2. Synthesis of Well-Aligned MWNTs

The synthesis of well-aligned MWNTs has been reported previously in [37,38] wherein a Ta plate was used as a substrate and a thin cobalt (Co) layer of 8 to 50 nm was coated by magnetron sputtering onto the surface of Ta substrate as catalyst. The nanotubes used have diameters of 80 to 120 nm and a length of about 10 μm depending on the Co layer thickness and growth time [37].

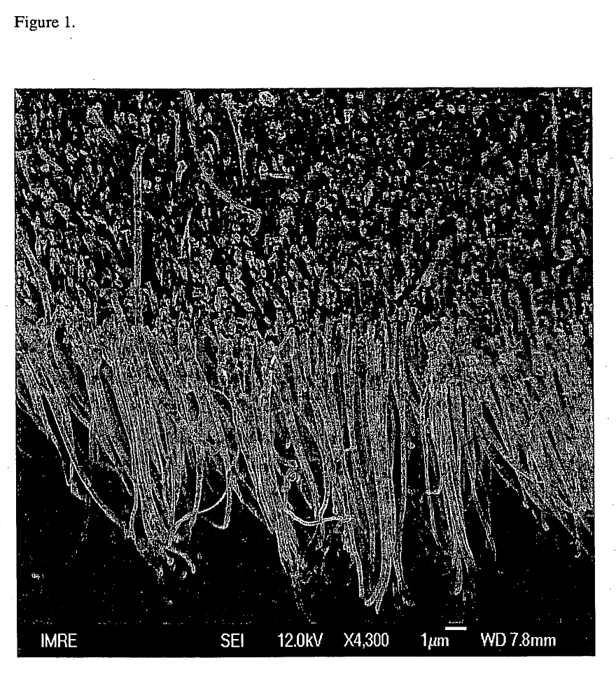

The typical morphology of the well-aligned carbon nanotubes is shown in FIG. 1. The Ta substrate with or without MWNTs was connected to the surface of a glassy carbon electrode by conductive silver paint (Structure probe, Inc., USA). The edge of the Ta substrate and glassy carbon electrode was insulated by pasting with nail enamel (Maybelline, NY, USA). These MWNTs connected to the glassy carbon electrode were used as the working electrode without any further treatment.

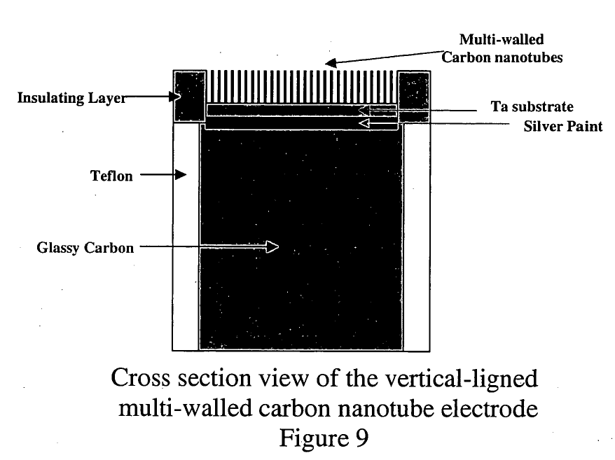

FIG. 9 is illustrative of a cross section of the vertical-aligned multi-walled carbon nanotube electrode and shows how the working electrode is constructed. The multi-walled carbon nanotubes are also depicted in FIG. 1.

7.3. Instrumentation

Voltammetric measurements were performed using CHI 660A electrochemical workstation (CH instruments Inc., USA) in a three-electrode arrangement, including a working electrode (MWNTs electrode, glassy carbon electrode or Ta substrate electrode), a platinum counter electrode and a 1 M KCl—Ag|AgCl reference electrode. All potentials were quoted versus the 1M KCl—Ag|AgCl reference electrode. All experiments were performed at room temperature (≈=25° C.). Differential pulse voltammetry (DPV) employed an increase potential of 4 mV, pulse amplitude of 5 mV, pulse period of 0.2 s and pulse width of 0.05 s.

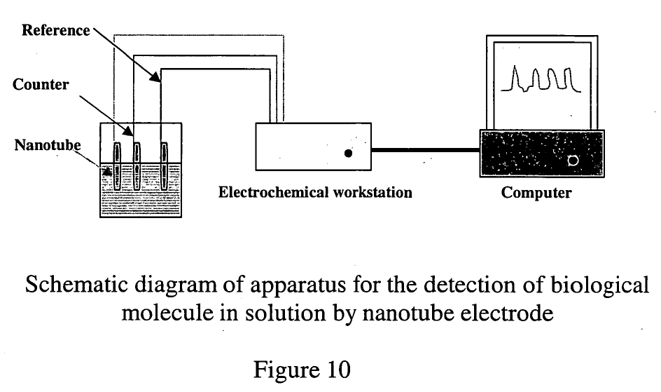

FIG. 10 is illustrative of the apparatus schematic showing the above three-electrode system, an electrochemical workstation and computer analysis means.

7.4. Procedure for UA Detection

Urine samples were obtained from laboratory co-workers. All urine samples were diluted with supporting electrolyte without further pretreatment before subject to voltammetric measurements. Standard addition method was employed for the determination of UA in the samples.

### RESULTS AND DISCUSSION

7.5. Cyclic Voltammetric Response of the MWNTs Electrode in PBS

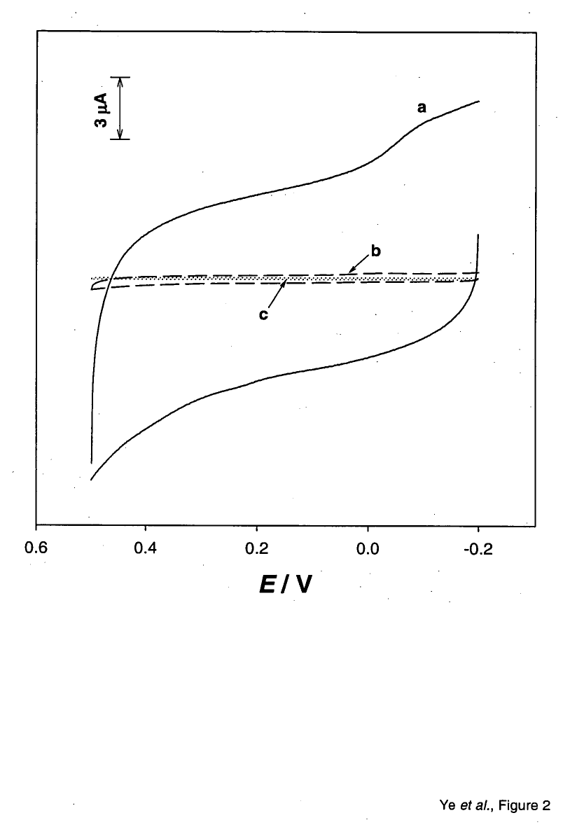

For electrochemical measurements, the Ta substrate with or without MWNTs was held against the surface of a glassy carbon electrode by silver paint. Typical voltammetric responses in PBS are shown in FIG. 2. Either the bare Ta substrate (FIG. 2, dotted line) or the glassy carbon electrode (FIG. 2, dashed line) gave a very small current response. However, when MWNTs electrode was used, the background current is significantly enhanced. In addition, the observed current is predominantly capacitive, since its magnitude depends linearly upon the voltage scanning rate.

The capacitance of an electrochemical apparatus depends on the separation between the charge on the electrode and the countercharge in the electrolyte. Since this separation is in nanometer scale for nanotubes electrodes, as compared with that of the micrometer or larger ordinary dielectric capacitors, very large capacitances can be resulted from the high nanotube surface area accessible to the electrolyte. The interfacial capacitance from the voltammetric responses is 0.64 mF·cm⁻² for the MWNTs electrode, which is much higher than that of the glassy carbon electrode or the bare Ta substrate electrode. This result implies that carbon nanotubes exhibit a very high specific capacitance [39] and can be used as an electrochemical apparatus.

7.6. Voltammetric Behavior of UA and L-AA at the MWNTs Electrode

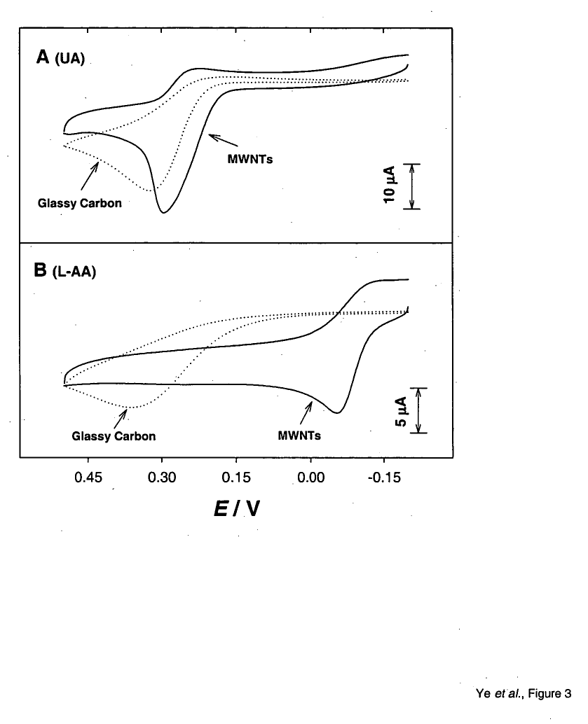

From the cyclic voltammogram of the MWNTs electrode in PBS (FIG. 2, solid line) we found no redox peaks between −0.20 V and +0.50 V. Thus, this MWNTs electrode provides a broad potential window for investigating the voltammetric behaviors of UA and L-AA. The cyclic voltammograms of UA and L-AA at either the glassy carbon or the MWNTs electrodes are shown in FIG. 3(A) and FIG. 3(B), respectively. As shown in FIG. 3(A), UA had a broad and relatively small CV peak response at about 0.323 V at the bare glassy carbon electrode, while the MWNTs displayed a sharp anodic peak at about 0.295 V with a slight increase in peak current as compared to that of glassy carbon electrode. The slight increase in peak current shows that the larger background current at the MWNTs electrode does not decrease the sensitivity for the detection of UA. In contrast, it can be seen from FIG. 3(B) that the oxidation of L-AA was broad and irreversible at about 0.357 V at the bare glassy carbon electrode. Of particular interest, at the MWNTs electrode, the peak potential shifted negatively to −0.058 V. The obvious decrease in the anodic overpotential to 0.295 V for UA and −0.058 V for L-AA shows a strong catalytic function of the MWNTs towards the oxidation of UA and L-AA. The shifts in the overpotentials may be due to a kinetic effect by which an increase in the rate of electron transfer from UA and L-AA to the MWNTs as observed in this experiment can be attributed to an improved reversibility of electron transfer processes on the MWNTs. Recently it was reported that MWNTs perform Nernstien behavior and fast electron-transfer kinetics for electrochemical reactions of Fe(CN)₆^{3-/4-} [40], suggesting a possibility of developing superior carbon electrodes based on MWNTs for electrochemical applications. The result in this study further confirms that MWNTs are superior materials for the construction of electrochemical sensors.

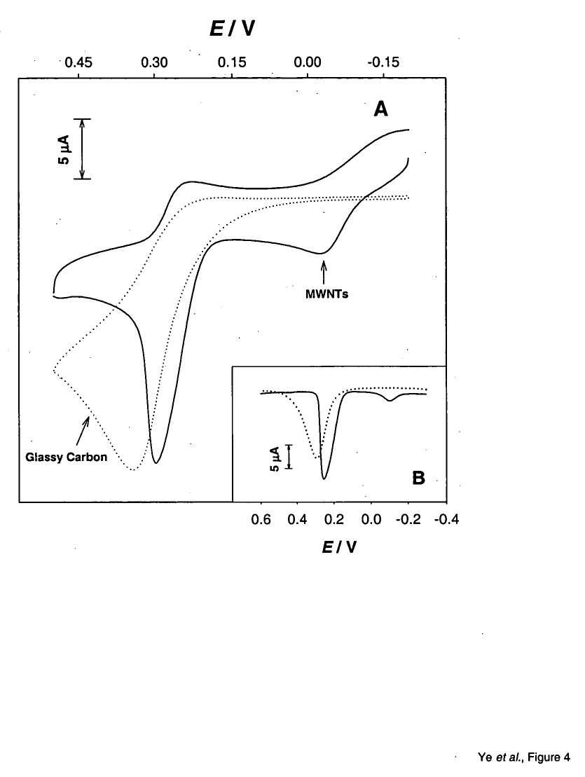

When 0.5 mM UA and 0.5 mM L-AA coexisted in the same sample, only an anodic peak was observed at about 0.342 V in CV (FIG. 4A, dotted line) and 0.325 V in DPV (FIG. 4B, dotted line) at bare glassy carbon electrode, and the peak potentials for UA and L-AA were indistinguishable. Thus it is impossible to separate UA from L-AA using the voltammetric peaks by the glassy carbon electrode. On the other hand, carbon nanotube electrode gave two well-defined voltammetric peaks at potentials of 0.297, −0.033 V in CV (FIG. 4A, solid line), and 0.253, −0.111 V in DPV (FIG. 4B, solid line), corresponding to the oxidation of UA and L-AA, respectively. The separations between the two peak potentials, about 0.330 V in CV and 0.364 V in DPV, are large enough for selective determination of UA in a mixture with L-AA. Both the density of MWNTs at Ta substrate and the exposed area of MWNTs to the electrolyte contribute to the current response of the MWNTs electrode. Hence, the current responses for the oxidation of UA and L-AA increase with the increase of the effective surface area of the MWNTs, while the potentials for the oxidation of UA and L-AA remain unchanged.

7.7. The Effect of pH on the Oxidation of UA and L-AA by the MWNTs Electrode

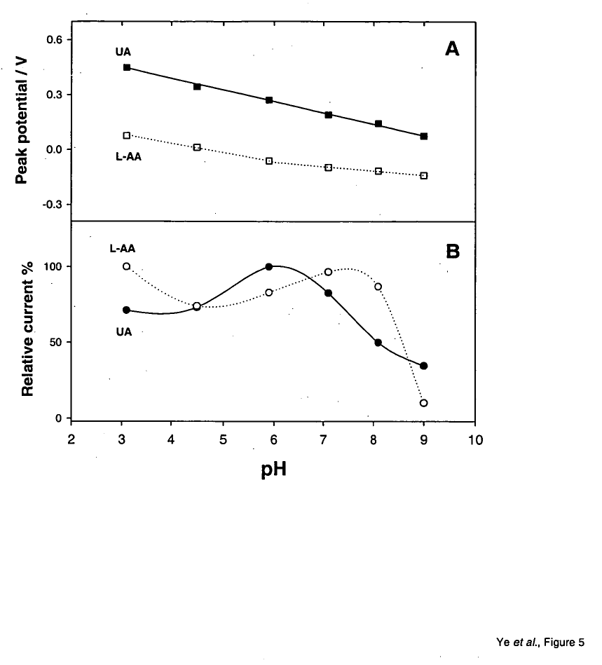

The effect of pH on the peak current and potential of the catalytic oxidation of UA and L-AA at the MWNTs were investigated using DPV, shown in FIG. 5. As can be seen in FIG. 5B, the current response reaches a maximum around pH 6.0 for UA, but a value of pH 7.5 for L-AA. The trend of the peak potentials (Eₚ) for both UA and L-AA shifting almost linearly towards negative potentials with an increase in pH (FIG. 5A) indicates that protons are directly involved in the rate determination step of the UA and L-AA oxidation reaction. The equations relating Eₚ (in volts) with pH, over the pH range of 3-9, is found to be: Eₚ=0.63−0.06 pH for UA with a correlation coefficient of 0.9983. The peak potential for UA shows a linear variation with pH with a slope of about −60 mV·pH⁻¹, which suggests that the total number of electrons and protons taking part in the charge transfer is the same in the tested pH range. For L-AA, the equations relating Eₚ (in volts) with pH are: (a) in pH 3.0-6.0, Eₚ=0.22−0.05 pH with a correlation coefficient of 0.9590 and (b) in pH 6.0-9.0, Eₚ=0.09−0.03 pH with a correlation coefficient of 0.9476. The slope of the Eₚ vs. pH is about 50 mV·pH⁻¹ between pH 3.0 and 6.0, indicating that a 1 e⁻/1H⁺ reaction is involved in the oxidation process, whereas at higher pH values the slope decreases to about 30 mV pH⁻¹, suggesting a 2 e⁻/1H⁺ transfer process. Consequently, the overall electrode reaction of L-AA at carbon nanotubes can be classified as an electrochemical reaction followed by a chemical reaction process as previously reported [7, 41]. Since the oxidation potential of UA is at least 270 mV more positive than that of L-AA in pH range 3.0-9.0, the simultaneous detection of UA and L-AA could be realized at the MWNT electrode in the tested pH range.

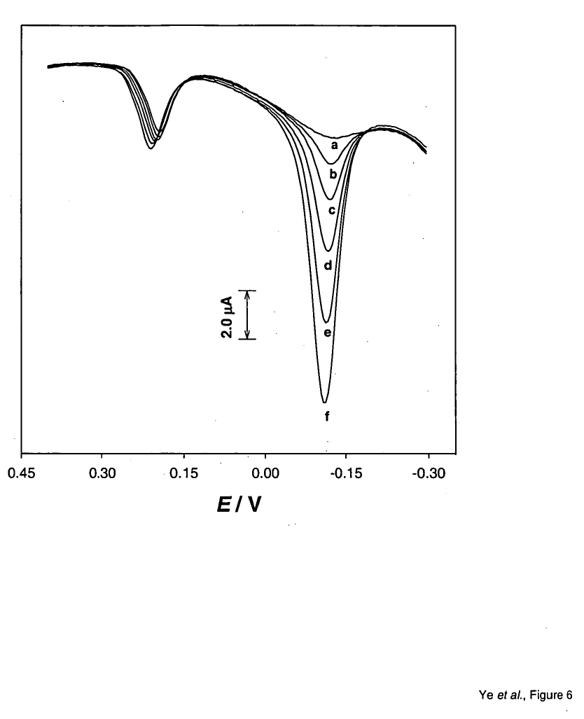

It is well known that L-AA coexists with UA in many samples [6-8]; therefore, its possible interference was investigated in a further detail in this study. FIG. 6 shows DPV responses of 5.0 μM UA in the presence of different concentrations of L-AA. The peak current for UA oxidation remains almost the same (2.9±0.1 AA) and the peak potential for UA oxidation shifts slightly from 190.9 mV to 212.7 mV when the concentration of L-AA increases from 0 to 400 μM. Furthermore, the current responses of UA and L-AA are separated with a potential difference of 400 mV as indicated in FIG. 5A; the MWNT actually responses much better to UA. It can also be seen in FIG. 6 that, although the detection of L-AA by the MWNT is not as sensitive as that of UA, simultaneous detection of UA and L-AA by the MWNT is achievable even with various L-AA concentrations. Since the acceptable tolerance of L-AA concentration for the detection of UA is presently demonstrated at least as much as 80-fold excess, the MWNT is expected to be applicable to various biological samples.

7.8. Linear Response and Reproducibility of the MWNTs Electrode

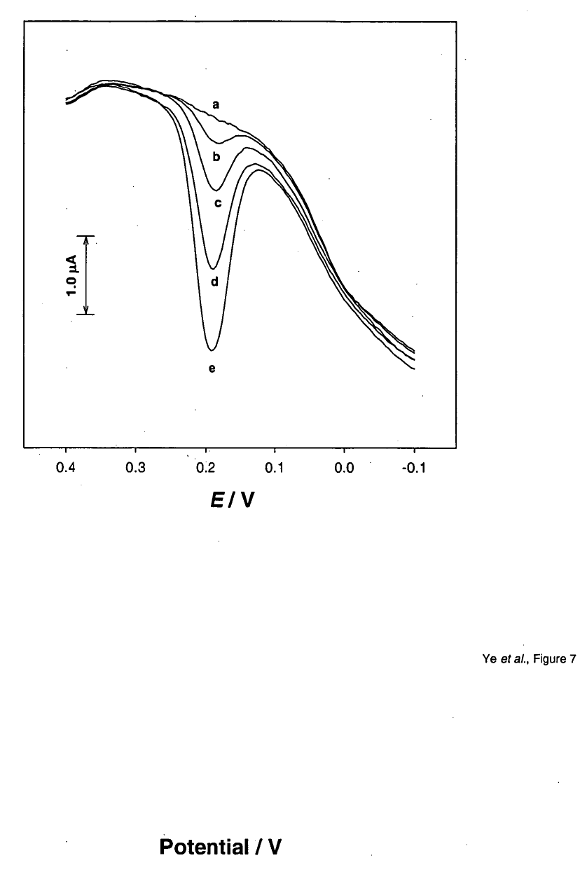

FIG. 7 shows the DPV response of UA at the MWNTs electrode in pH 7.4 PBS with concentration of (a) 0, (b) 0.5, (c) 1.0, (d) 2.0 and (e) 4.0 μM. The DPV current response is linearly dependent on the concentration of UA between 0.2 μM and 80 μM with a slope (μA/μM) and correlation coefficient of 0.61 and 0.9997, respectively. The detection limit (3δ) is 0.1 μM. Furthermore, the reproducibility of the MWNT for the determination of UA was investigated. Repetitive measurements were carried out in the solution of 5.0 μM UA in pH 7.4 PBS. The results of 9 successive measurements gave a relative standard deviation of 2.3% (data not shown).

UA can be easily adsorbed at different electrodes such as carbon paste electrode, activated glassy carbon electrode and platinum electrode [3, 5, 13, 14]. To test if the adsorption of UA took place at the surface of MWNTs, the MWNTs electrode was soaked in 50 μM UA with pH 7.4 PBS for 24 hrs. Then, it was taken out and washed using pH 7.4 PBS. The electrode was dipped in blank PBS and the DPV response for the UA adsorbed at the MWNTs was examined. No current response for UA oxidation was detected (data not shown), indicating that (a) UA will not be absorbed at the MWNTs and (b) the MWNTs electrode can be reused for continuous detection of UA by simple cleaning the surface.

7.9. Detection of UA in Human Urine Sample

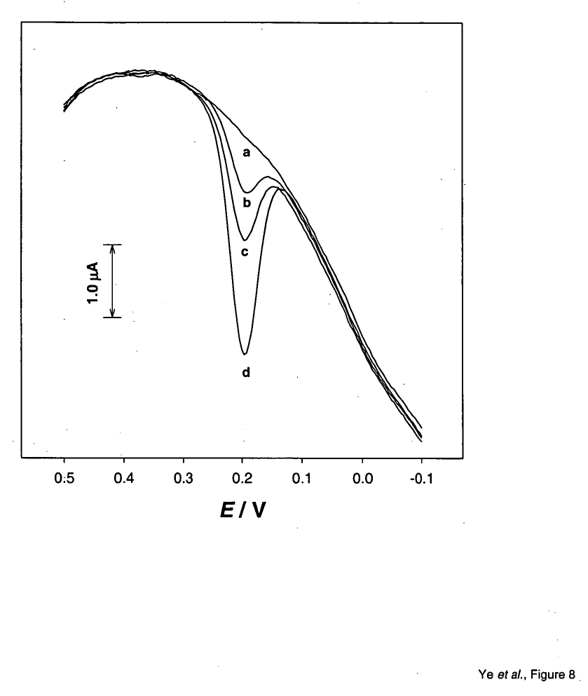

Human urine samples from laboratory co-workers were determined at well-aligned MWNTs electrode. To fit into the linear range, the samples were diluted by 250 or 500 times with PBS before analysis without other pretreatments. Standard addition method was employed. FIG. 8 shows a typical DPV response of a (a) blank PBS, and urine sample diluted (b) 500 times, (c) 250 times and (d) 250 times with addition of 2.0 μM UA. The recovery determined by spiking the samples with a measured amount of standard UA was found to be between 95.3% and 105.5%. Additionally, UA concentrations found in urine samples by the MWNTs electrode in the range of 450 μM and 1250 μM are fairly close to those reported elsewhere [13-22].

In accordance with this invention, the selective voltammetric detection of uric acid from a blood or urine sample even in the presence of ascorbic acid is now made possible through the innovative use of a high-density, well-aligned, carbon nanotube electrode in an electrochemical apparatus.

The invention may also broadly be said to consist in the parts, elements and features referred to or indicated in the specification of the application, individually or collectively, and any or all combinations of any two or more of the parts, elements or features and where specific integers are mentioned herein which have known equivalents such equivalents are deemed to be incorporated herein as if individually set forth.

### 8. MODIFICATIONS OF THE PREFERRED EMBODIMENTS

While the invention has been described with particular reference to certain embodiments thereof, it will be understood that various modifications can be made to the above-mentioned embodiment without departing from the spirit and scope of the present invention. The examples and the particular proportions set forth are intended to be illustrative only.

The skilled reader will instantly realize that, although the Examples have been limited to the detection of Uric Acid from a biological fluid such as blood or urine that without departing from the scope of the invention other biological acids and like molecules may also be selectively detected using the protocols set forth.

The present invention enables previously difficult to detect molecules to now be measured by the innovative use of carbon nanotube technology and whereby high-density, well-aligned carbon nanotubes are used as electrochemical sensors. As a consequence, the inventors also contemplate that included within the scope of the invention will be the use of carbon nanontubes, and in particular MWNT's as electrodes in an electro-chemical diagnostic, analytical,or forensic test.

The skilled reader will also understand that depending on the molecule to be detected the concentrations of components may vary as may the electrical parameters at which the assay is run.

Throughout the description and claims of this specification the word “comprise” and variations of the word, such as “comprises” and “comprising”, are not intended to exclude other additives, components, integers or steps

### 9. ACKNOWLEDGEMENTS

This inventive work was supported by an Academic Research Grant from the National University of Singapore R-377-000-015-112 to F.-S. S.

### 10. REFERENCES

[1] V. S. E. Dutt, H. A. Mottola, Anal. Chem. 1974, 46, 1777.
[2] J. P. Hart, Electroanalysis of Biologically Important Compounds, Ellis Horwood, Chichester 1990, pp. 5 1.
[3] J. M. Zen, J. S. Tang, Anal. Chem. 1995, 67, 1892.
[4] R. N. Adams, Anal. Chem. 1976, 48, 1120A.
[5] H. Ernst, M. Knoll, Anal. Chim. Acta 2001, 449, 129.
[6] J. A. Stamford, Jr. J. B. Justice, Anal. Chem. 1996, 68, 359A.
[7] L. Zhang, X. Lin, Analyst 2001, 126, 367.
[8] P. Capella, B. Ghasemzadeh, K. Mitchell, R. N. Adams, Electroanalysis 1990, 2, 175.
[9] J. M. Zen, C. T. Hsu, Talanta 1998, 46, 1363.
[10] J. M. Zen, J. J. Jou, G. Ilangovan, Analyst 1998, 123, 1345.
[11] Z. Gao, K. S. Siow, A. Ng, Y. Zhang, Anal. Chim. Acta 1997, 343, 49.
[12] Z. Gao, H. Huang, Chem. Commun. 1998, 19, 2107.
[13] J. Wang, M. S. Lin, Electroanalysis 1990, 2, 861.
[14] K. Shi, K. K. Shiu, Electroanalysis 2001, 13, 1319.
[15] Z. Gao, J. Electroanal. Chem. 1994, 365, 197.
[16] J. M. Zen, Y. J. Cheng, C. T. Hsu, Y. S. Ting, Electroanalysis 1997, 9, 1009.
[17] F. E. Keedy, P. Vadgama, Biosens. Bioelectron. 1991, 6, 491.
[18] E. Gonzalez, F. Pariente, E. Lorenzo, L. Hernadez, Anal. Chim. Acta 1991, 242, 267.
[19] M. J. Rocheleau, W. C. Purdy, Electroanalysis 1991, 3, 935.
[20] P. M. Ajayan, Chem. Rev. 1999, 99, 1787.
[21] R. Saito, M. Fujita, G. Dresselhaus, M. S. Dresselhaus, Appl. Phys. Lett. 1992, 60, 2204.
[22] J. W. G. Wildoer, L. C. Venema, A. G. Rinzler, R. E. Smalley, C. Dekker, Nature 1998, 391, 59.
[23] T. W. Odom, J. L. Huang, P. Kim, C. M. Lieber, Nature 1998, 391, 62.
[24] P. J. Britto, K. S. V. Santhanam, P. M. Ajayan, Bioelectrochem. Bioenerg. 1996, 41, 121.
[25] E. Pennisi, Sci. News 1992, 142, 36.
[26] Z. H. Wang, J. Liu, Q. L. Liang, Y. M. Wang, G. Luo, Analyst 2002, 127, 653.
[27] J. J. Davis, R. J. Coles, H. A. O. Hill, J. Electroanal. Chem. 1997, 440, 279.
[28] H. Luo, Z. Shi, N. Li, Z. Gu, Q. Zhuang, Anal. Chem. 2001, 73, 915.
[29] P. J. Britto, K. S. V. Santhanam, A. Rubio, J. A. Alonso, P. M Ajayan, Adv. Mater. 1999, 11, 107.
[30] J. Kong, N. R. Franklin, C. Zhou, M. G. hapline, S. Peng, K. Cho, H. Dai, Science 2000, 287, 622.
[31] P. G. Collins, K. Bradley, M. Ishigami, A. Zettl, Science 2000, 287, 1801.
[32] J. N. Barisci, G. G. Wallace, R. H. Baughman, J. Electroanal. Chem. 2000, 488, 92.
[33] C. Y. Liu, A. J. Bard, F. Wudl, Electrochem. Solid State Lett. 1999, 2, 577.
[34] C. Niu, E. K. Sichel, R. Hoch, D. Moy, H. Tennet, Appl. Phys. Lett. 1997, 70, 1480.
[35] H. Luo, Z. Shi, N. Li, Z. Gu, Q. Zhuang, Chem. J. Chin. Univ. 2000, 21, 1372.
[36] P. Liu, J. Hu, Sensors and Actuators B 2002, 84, 194.
[37] W. D. Zhang, Y. Wen, S. M. Lin, W. C. Tjiu, G. Q. Xu, L. M. Gan, Carbon, 2002, 40, 1981.
[38] W. D. Zhang, Y. Wen, W. C. Tjiu, G. Q. Xu, L. M. Gan, Appl. Phys. A 2002, 74, 419.
[39] F. Marken, M. L. Gerrard, I. M. Mellor, R. J. Mortimer, C. E. Madden, S. Fletcher, K. Holt, J. S. Fpprd, R. H. Dahm, F. Page, Electrochem. Commun. 2001, 3, 177.
[40] J. M. Nugent, K. S. V. Santhanam, A. Rubio, P. M. Ajayan, Nano Lett. 2001, 1, 87.
[41] I. F. Hu, T. Kuwana, Anal. Chem., 1986, 58, 3235.

1. An electrochemical method for detecting the presence of one or more molecules of interest in a sample, wherein the method comprises a voltammetric analysis step using as a working electrode a coated or layered electrode such as a carbon nanotube electrode, a glassy carbon electrode or a Ta substrate electrode and wherein the electrode has a reduced overpotential.

2. An electrochemical method according to claim 1 for selectively detecting the presence of one or more molecules of interest from a biological sample, wherein a carbon nanotube electrode is used as the working electrode.

3. A method according to claim 1 wherein, the working electrode comprises a plurality of carbon nanotubes.

4. A method according to claim 3 wherein, the plurality of carbon nanotubes comprising the working electrode is/are high-density, multi-walled, vertically-aligned carbon nanotubes (MWNT's).

5. A method according to claim 4 wherein, the MWNT's are vertically aligned on a substrate.

6. A method according to claim 5 wherein, the substrate is a plate comprising a Ta, Co, Ni, V, Nb, Db, Pd, W, Mo, Cu, Fe, Si, Au, Pt, stainless steel, glassy carbon, graphite and diamond, or a mixture thereof.

7. A method according to claim 4 wherein, the high-density, multi-walled, vertically-aligned carbon nanotubes aligned on the substrate act as electrochemical sensors.

8. A method according to claim 1 wherein, the voltammetric analysis step allows for the selective detection of Uric Acid (UA) in the presence of L-Ascorbic Acid (L-AA) in a sample of interest.

9. A method according to claim 8 wherein, the L-Ascorbic Acid (L-AA) concentration is at least as high as 400 μM or within standard acceptable tolerance levels.

10. A method according to claim 1 wherein, the voltammetric analysis step allows for a voltammetric measurement in a cyclic voltammetry mode or a differential pulse voltammetry mode.

11. A method according to claim 10 wherein, the differential pulse voltammetry mode employs an increased potential of about 4 mV, a pulse amplitude of about 5 mV, a pulse period of about 0.2 s and a pulse width of about 0.05 s.

12. A method according to any claim 1 wherein, the sample of interest is a biological fluid.

13. A method method according to claim 1 wherein the sample of interest is a biological fluid selected from blood, urine, sweat, saliva, plasma, spinal fluid, embryionic fluid, brain tissue, a cell culture, a tissue culture and a mixture thereof.

14. A method according to claim 13 wherein, the biological fluid is a human blood or a human urine sample prepared by a simple dilution without the need for further pretreatment.

15. A method according to claim 1 wherein, the one or more biological molecules of interest is Uric Acid (UA) and/or L-Ascorbic Acid (L-AA).

16. A method according to claim 1 wherein, the nanotube (MWNT's) electrode acts as electrochemical sensors sensitive to the presence of Uric Acid (UA) and L-Asorbic Acid (L-AA).

17. A method according to any one of the claim 1 wherein, the concentration of the molecule of interest is from 1.0×10⁻¹ to 1.0×10⁻⁹ mol/L, and more preferably from 1.0×10⁻³ to 1.0×10⁻⁷ mol/L

18. An apparatus for use in a method according to claim 1 for electrochemically detecting the presence of one or more molecules of interest in a sample, the apparatus comprising the following operably connected components:
(i) an electrochemical means,
(ii) an analysis means, and
(iii) a voltammetric sensing means,
wherein the voltammetric sensing means is a three-electrode arrangement comprising:
a working electrode having a high-density, multi-walled,
vertically-aligned carbon nanotubes (MWNT's) electrode, a platinum counter electrode and
a 1M KCl—Ag|AgCl reference electrode.

19. An apparatus according to claim 18 wherein, the working electrode comprises high-density, multi-walled, vertically-aligned carbon nanotubes (MWNT's) connected to a glassy electrode via a Ta substrate.

20. An apparatus according to claim 18 for use in a biological assay testing for the presence of a biological acid in a sample.

21. An apparatus according to claim 18 wherein the assay is designed to detect the presence of Uric Acid in a blood or urine sample.

22. A use of a high density, multi-walled, vertically-aligned, carbon nanotube (MWNT) in a method according to claim 1.
